(19) United States
(12) Patent Application Publication
(10) Pub. No.: US 2013/0116496 A1
(43) Pub. Date: May 9, 2013

(54) APPARATUS AND METHOD FOR RADIATION TREATMENT OF A DESIRED AREA IN THE RENAL VASCULAR SYSTEM OF A PATIENT

(71) Applicant: RON WAKSMAN, POTOMAC, MD (US)

(72) Inventor: RON WAKSMAN, POTOMAC, MD (US)

(73) Assignee: RON WAKSMAN

(21) Appl. No.: 13/289,740

(22) Filed: Nov. 4, 2011

(51) Int. Cl. A61N 5/00

(52) U.S. Cl. 600/1

(57) ABSTRACT

According to another general aspect, an apparatus for irradiating a plurality of renal nerves comprising a guidewire used to lead an elongated catheter in the human body; the enlongated catheter further comprising a plurality of lumens; the plurality of lumens further comprising a source train lumen, a hydraulic pump lumen, a guidewire lumen, and an inflator lumen; the elongated catheter is connected to the guidewire by inserting the guidewire into the guidewire lumen; the elongated catheter contains a source train within the source train lumen; the source train lumen and hydraulic pump lumen are used to push the source train to a renal vessel.

### CROSS REFERENCE TO RELATED APPLICATION

N/A

### FIELD OF THE INVENTION

The invention generally relates for beta radiation treatment of a desired area in the vascular system to accomplish catheter-based administration of ionizing (beta) radiation in or near the renal artery, or near the renal sympathetic nerve to treat hypertension or any other medical condition.

### BACKGROUND

Hypertension, also known as high blood pressure, is a common cardiovascular disease that affects millions of individuals worldwide. Hypertension is a disease where the blood pressure refers to the force of blood pushing against the artery walls as it courses through the body. Like air in a balloon and water in a rubber pipe, the blood of the body fills arteries to a certain capacity. The pressure of the blood can exceed the normal levels of the walls of the arteries, and just like too much air in a balloon that results in the balloon to explode or burst, the same results occur in the human body. Hypertension is the leading cause of stroke and a major cause of heart attacks.

Hypertension is diagnosed by getting a local check up, when a nurse or doctor measures the blood pressure. The blood pressure readings appear with two numbers. The first and higher of the two is a measure of systolic pressure and the pressure in the arteries when the heart beats and fills them with blood. The second number measures diastolic pressure or the pressure in the arteries when the heart rests between beats. The normal pressure is 120/80 in healthy adults. Many people with the high blood pressure do not realize that the disease is affecting them. Thus, hypertension is called the “silent killer” because it rarely causes symptoms, and even as it inflicts serious injury to the body. If left untreated, hypertension can cause vision problems, heart attack, stroke, and kidney failure.

Currently, the treatment goal would be reducing hypertension. The conventional treatment method is to provide medicine such as alpha blockers, Angiotensin-converting enzyme (ACE) inhibitors, Angiotensin receptor blockers (ARBs), Beta blockers, Calcium channel blockers, Central alpha agonists, Diuretics, Renin inhibitors, including aliskiren (Tekturna), and Vasodilators. However, taking one or more of these drugs alone may not be enough to control hypertension. There must also be a form of controlling ones diet as well eating a heart-healthy diet, including potassium and fiber, and drink plenty of water, exercise regularly—at least 30 minutes a day, if individuals smoke, the individuals must stop, limit how much alcohol individuals drink—1 drink a day for women, 2 a day for men, limit the amount of sodium (salt) individuals eat—aiming for less than 1,500 mg per day, individuals should try to reduce stress, individuals can also try meditation or yoga, and stay at a healthy body weight; the treatment options may still not control an individual's hypertension. Even with all these measures available, these methods may not provide a viable treatment option to control hypertension.

Another treatment option that is available is renal denervation. Currently, Adrian, which is owned by Medtronic, uses a RF radio-frequency ablation device to treat hypertension. However, there are many problems with this modality since RF electrode requires multiple locations of high-frequency electric current within the human body. By having multiple location burns, there is a very high likelihood of unintended burns to the artery or skin that will result into unnecessary damage. An unintended burn can result into complications for the patient that will not be recognized immediately, but only after months and months continuous check up. Careful stepping and direct tissue contact is required for precise positioning of RF probes to avoid excessive burns results in lengthy dwell times that generally increase complications for intravascular procedures. Analgesics and more powerful drugs, such as morphine, for pain management are also required for RF burning which add risks to the procedure. In addition, RF ablation creates burns with charring and formation of thrombus which will obstruct blood flow to the kidneys resulting in renal failure. Therefore, there is a need to have a vascular brachytherapy system that allows for rapid delivery of limited range ionizing radiation, such as beta radiation from a radioactive source, to create lesions for renal denervation to treat hypertension.

Therefore, one of ordinary skill in the art would appreciate a system that provides an effective treatment for hypertension without causing tissue damage or other complication to the patient.

### SUMMARY OF INVENTION

According to one general aspect, there is a a method of irradiating a plurality of renal nerves by puncturing a human body to insert a guidewire to a region of interest; connecting the guidewire to an elongated catheter distal end portion into the circulatory system of the human body; inserting the elongated catheter into the human body until the elongated catheter distal end is located near a renal vessel; adjusting the elongated catheter distal end near a tissue wall of the renal vessel, wherein, the tissue wall of the renal vessel is in close proximity to the plurality of renal nerves; moving a source train through a lumen within the elongated catheter until the source train reaches the renal vessel; irradiating the plurality of renal nerves by holding the source train for a predetermined time interval in the renal vessel; and removing the source train from the renal vessel after a lapsed in the predetermined time interval. Further, in the method of the guidewire can be shaped comprises a straight distal end, a pigtail shape distal end, or a half circle distal end. In addition, the method of said elongated catheter distal end may expand or shrink; the method further comprising inflating an inflator by inserting gas or liquid into a inflator lumen.

According to another general aspect, an apparatus for irradiating a plurality of renal nerves comprising a guidewire used to lead an elongated catheter in the human body; the enlongated catheter further comprising a plurality of lumens; the plurality of lumens further comprising a source train lumen, a hydraulic pump lumen, a guidewire lumen, and an inflator lumen; the elongated catheter is connected to the guidewire by inserting the guidewire into the guidewire lumen; the elongated catheter contains a source train within the source train lumen; the source train lumen and hydraulic pump lumen are used to push the source train to a renal vessel. Further, the apparatus for irradiating the plurality of renal nerves comprising the elongated catheter at the distal end further comprising an inflator, wherein the inflator is connected to the inflator lumen and the inflator lumen can expand and contract depending on the fill volume. In addition, the apparatus for irradiating the plurality of renal nerves comprising the inflator at the distal tip of the elongated catheter is located inside the wrapped the elongated catheter; wherein, the elongated catheter can expand to the renal vessel walls.

### DESCRIPTION OF THE DRAWINGS

FIG. 1 illustrates an overview of the human body organs.

FIG. 2 illustrates a catheter based system to be inserted into the human body.

FIG. 3 illustrates an overview of the human kidneys, with left side irradiated.

FIG. 4 illustrates an overview of the human kidneys, with right side irradiated.

FIG. 5 illustrates an overview of the human kidneys, with left side irradiated with pigtail shape.

FIG. 6 illustrates an overview of the human kidneys, with right side irradiated with pigtail shape.

FIG. 7 illustrates a source train within the system.

FIG. 8 illustrates the human circulatory in which the catheter is inserted into the human body and traveled to the renal area, but not limited to this method.

FIG. 9 illustrates an overview of the human kidneys, with left side irradiated with pigtail shape expanded with an inflator.

FIG. 10 illustrates a source train as it moves through the catheter.

FIG. 11 illustrates a view of the renal vessel with a pigtail shape expanded with an inflator.

FIG. 12 illustrates a structural set of lumens, but not limited to that set, within the catheter.

While the invention will be described in connection with the preferred embodiment, it will be understood that it is not intended to limit the invention to that embodiment. On the contrary, it is intended to cover all alternatives, modifications, and equivalents, as may be included within the spirit and scope of the invention as to be defined by claims to be filed in a non-provisional application.

### DETAILED DESCRIPTION

The invention generally relates to an apparatus and method for beta radiation treatment of a desired area in the vascular system. The goal of therapy is to influence renal function. Therapy is accomplished by catheter-based administration of ionizing (beta) radiation in or near the renal artery, or near the renal sympathetic nerve to modulate functions of the nephron, the vasculature, and the renin-containing juxtaglomerular granular cells and nerve conduction.

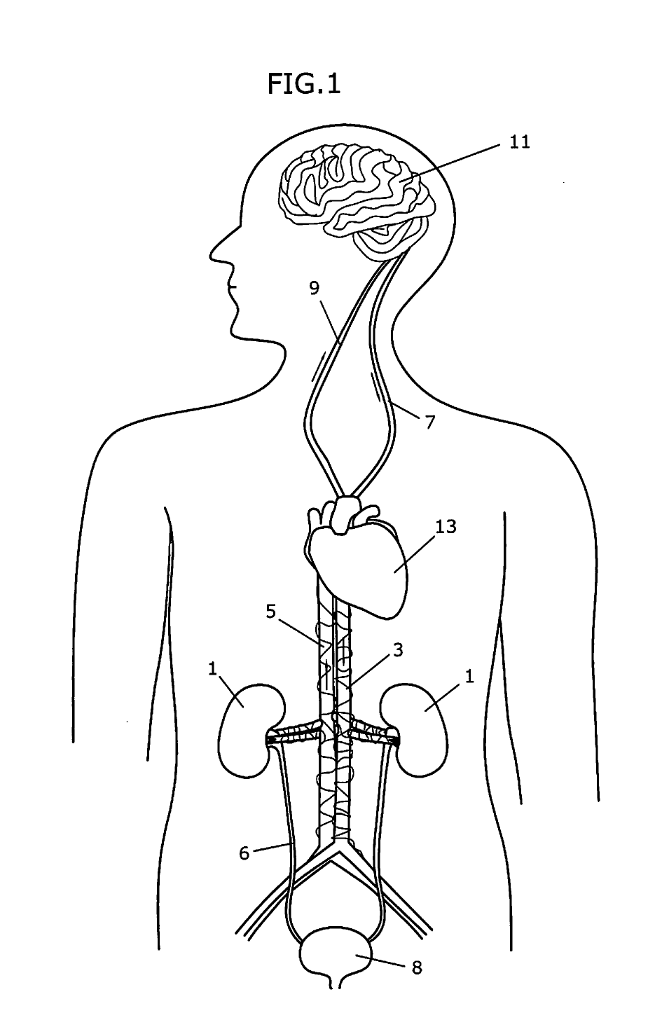

FIG. 1 shows a general diagrammatical view of the human body with organs within it. To better understand the present invention, it is helpful to have at least a basic introduction of the physiology of the kidney and to the renal sympathetic afferent and efferent nerve activity. Initially, the brain 11 will send information to renal efferent nerve 7 that will result in rennin release, the control of sodium retention and the renal blood flow. The kidneys 1 will process all the fluids in the human blood and will remove waste depending on the message from the brain. This is a simple physical process. Water, salts and other substances are filtered through the membranes from blood plasma holding back formed elements of the blood and large proteins inside the kidneys. The waste in the blood is secreted through the right and left ureter 6 to the gallbladder 8. The kidney 1 then will send renal afferent information back to the brain 11. The brain 11 processes this information and starts to control and change the beat of the heart 13 to determine if enough oxygen consumption is supplied. If the body starts to retain large amounts of sodium the blood flows from the anterior tibial artery, peroneal artery, and posterior tibial artery become harder to flow through. If enough oxygen is not supplied, the brain 11 tells the heart 13 to pump harder and faster, which can result into a heart attack. In addition, if there is not enough oxygen in the blood, the brain may suffer from a stroke. These organs aide in the pressure for hypertension. However, there is a new procedure that can help in controlling hypertension. The procedure of inserting a beta radiation emitting catheter into the human body to irradiate the renal vessel and nerve around the veins 3 and arteries 5 of the kidneys; the blocking of the brain signal back and forth to the kidney, this Renal Nerve Radiation (RNR) quiets the renal nerves is countering the conic activation of the sympathetic nerve system and provides a sustaining reduction in both blood pressure and the amount of damaging neuro-blood hormones.

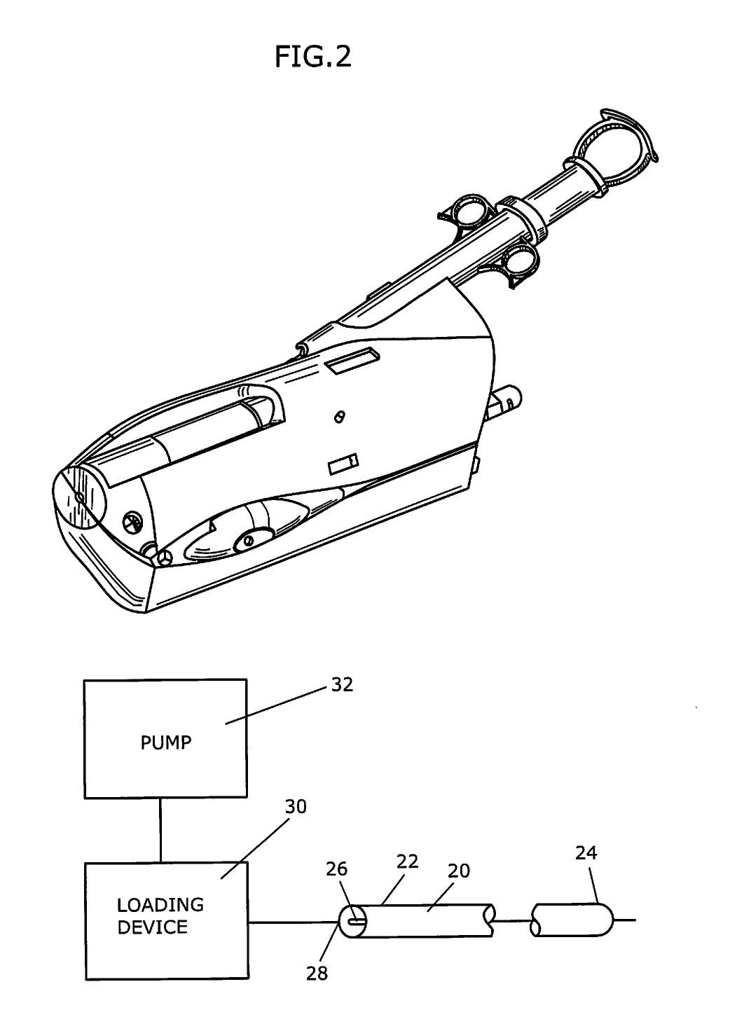

FIG. 2 depicts the Best Vascular System that may be employed in the present invention in general diagrammatic form for ease of initial understanding. Shown in FIG. 2 is an elongated catheter 20 having a proximal end portion 22, a distal end portion 24, and at least one source or send lumen 26 extending there between. The catheter is sized for insertion of the distal end portion through the vascular system of a patient to a selected area in the renal artery to be ablated, such as the AV node or other site. This may be carried out, for example, by inserting the catheter percutaneously and advancing the catheter over a typical guide wire 28 into the aorta and/or the vena cava via transseptal puncture and catheterization. Guide wires and procedures used in advancing such a catheter to the point of treatment are well known and will not be discussed in detail.

Further in FIG. 2, at the proximal end of the catheter, which is located outside the patient in a percutaneous procedure such as described above, a transporting and/or loading device 30 is provided for loading a radioactive source or train of sources, such as pellets or capsules (also called “seeds”) comprising or containing radioactive material, into the send lumen 26 of the catheter 20. Additional seeds may also be loaded such that the total length of the combined seeds corresponds to at least the length of the lesion to be ablated.

After the radioactive source or source train is loaded, pressurized blood-compatible liquid, such as sterile saline solution or sterile water, is introduced via liquid source 32 through a port 34 in the proximal end of the send lumen 26 behind the source(s). Flow of liquid through the lumen pushes the source(s) along the send lumen to the distal end portion, which is located at the site to be treated. The liquid which provides the motive force for moving the sources may be allowed to exit from the distal end of the catheter, but is preferably returned in a parallel return lumen provided in the catheter that communicates at the distal end of the catheter with the send lumen.

After the radioactive source or sources train is located at the desired site, it is allowed to remain for a time sufficient to irradiate the tissue. It is apparent that the source train, although made up of separate radioactive seeds or pellets, provides an elongated and essentially continuous radiation source that may be used to form lines of irradiated tissue through the arteries of and to kidney. The radioactive sources are preferably beta-emitting, although gamma-emitting, x-ray or other sources could be used, and the residence time period will be relatively short, on the order of minutes as discussed in more detail below. The activity of the radiation sources and the residence time may vary and be selected depending on the thickness of the renal artery tissue to be irradiated. The precise activity and residence time is presently not fully known, but may be ascertained with routine and well know testing techniques that do not require undue experimentation.

Vascular brachytherapy allows for the delivery of ionizing radiation from the inside the vasculature to yield short dwell times of less than 5 minutes which also minimizing radiation injury to critical non-target tissues. With vascular brachytherapy treatment of arteries, radiation is delivered to the adventitia to prevent hyperlasia, i.e., the proliferation of cells, after interventions that damage the vessel wall to restore patency for adequate blood flow. In the case of the Novoste Beta-Cath System, a single treatment plan requires placement of delivery catheter, a choice of radioactive source length, assessment of vessel diameter for dwell time, followed by hydraulic delivery of the source, irradiation dwell (usually less than 5 minutes) and hydraulic return of the source to the storage device. A variant of the Beta-Cath System with a catheter shaped to fit the renal vessel can be used to deliver ionizing beta radiation to the renal artery and associated nerves and without the complications, charring, thrombus or pain associated with arterial RF ablation burns, to control hypertension. The Novoste Beta-Cath System has been used successfully to create transmural lesions blocking nerve conduction in the cavotricuspid isthmus of canines. Ionizing radiation presents several advantages over RF ablation and other modalities for the treatment of hypertension. Ionizing radiation can create a nerve conduction block with a single dwell position, without displacement or repositioning of the treatment catheter and without the requirement of being in direct contact with the renal vessel wall. In addition, the lesion and subsequent denervation is created over time by multiple several mechanisms, including gradual fibrotic replacement of the underlying capillaries and has the added benefit of avoiding endothelial disruption and thrombus on the vessel surface. Also, the very small diameter radioactive sources and delivery catheters used for vascular brachytherapy, presently 3.5 F (1.17 mm), are able to treat small diameter renal vessels that are not accessible or treatable by large RF ablation probes. Thirty percent of patients have accessory or anomalous renal vessels which may require treatment for hypertension. The ability of vascular brachytherapy to deliver the required amount of energy, rapidly, thoroughly and precisely to tissues that are inaccessible to RF technologies is novel.

After the treatment is complete, the catheter may be removed or shifted to a different treatment position. The radioactive sources are preferably returned to the leading device while the catheter is removed or shifted in order to avoid undue radiation exposure to the patient. To retrieve the radioactive sources, liquid may be forced through the send lumen in a reverse direction to return the treating element to the proximal end and into the loading device, if desired, before removal of the catheter. The reverse flow of fluid may be achieved by forcing liquid under positive pressure through the return lumen in a reverse direction, which forms a closed loop with the send lumen, forcing the sources in a reverse direction to the loading device 30.

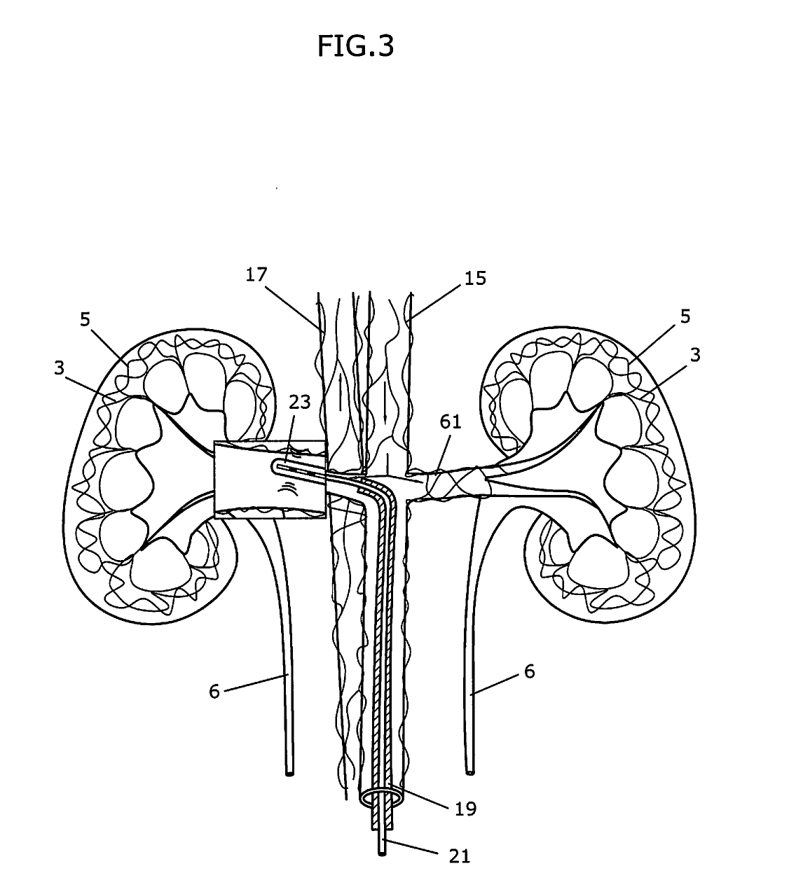

FIGS. 3, 4, 5, and 6 illustrate the present invention employing a guide wire 21 with a preformed tip, shaped as desired, e.g. curved to conform to the wall of the renal artery to be irradiated, to provide an active positioning means for the catheter. As shown in FIG. 3, the guide wire 21 is first inserted through a guide tube or sheath 19 into the right artery, preferably through the Inferior Vena Cava 17, where it is positioned against the renal wall at the location to be irradiated (which may be identified by a procedure called mapping).

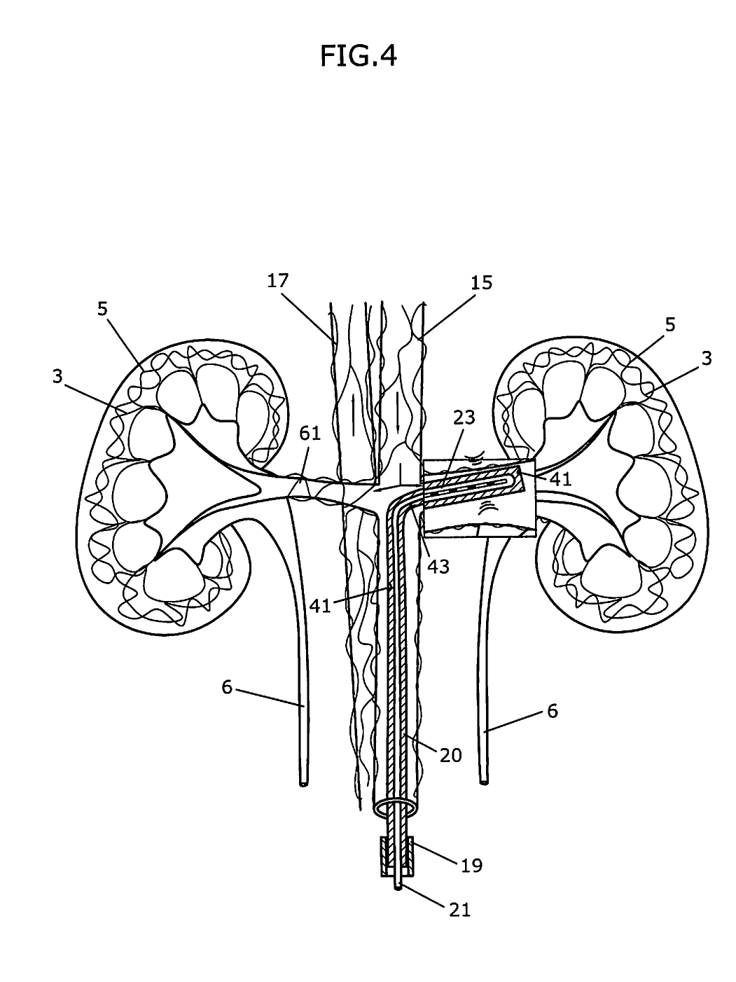

As seen in FIG. 4, the catheter 30 in accordance with the present invention is advanced over the guide wire 21, through the guide tube or sheath 19, until it lies along the surface of the renal vein 17 to be irradiated. After it is inserted to the proper location, the train of radioactive seeds is advanced (as by hydraulic force) to the distal end 23 of the catheter, which lies against the renal wall. The radioactive seed train is allowed to remain in the distal end until sufficient radiation dose is provided to induce a lesion along the line where the distal end of the catheter lies. The radioactive seed train may then be retrieved for repositioning or removal of the catheter. Because the radiation sources are not located in the catheter during introduction, positioning or withdrawal, overexposure of the vasculature to radiation is minimized.

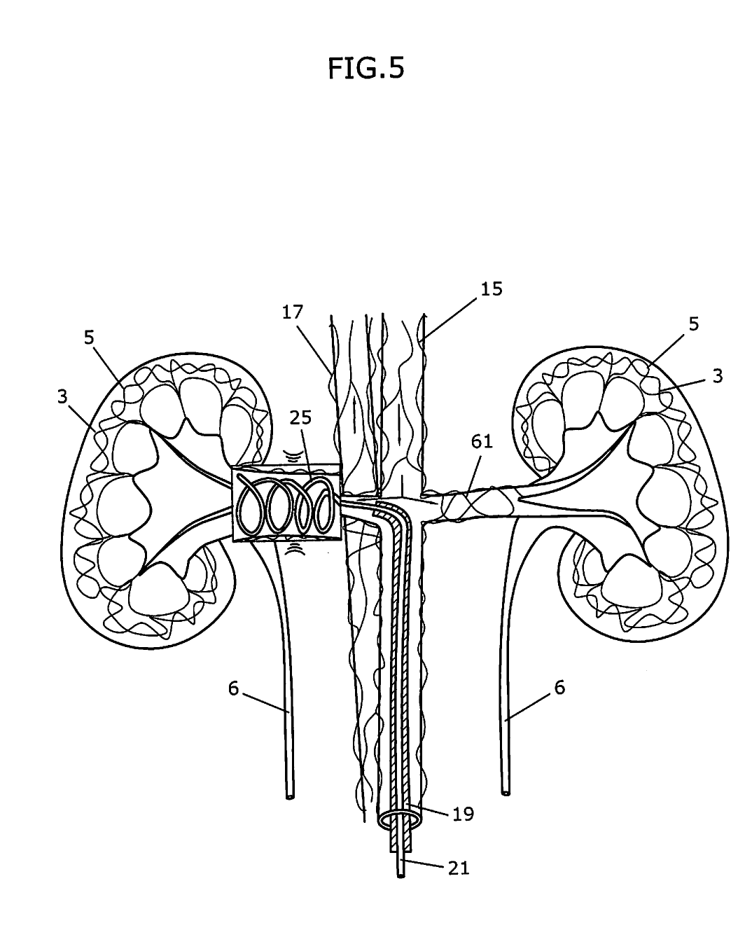

FIG. 5 illustrates, similar to FIG. 3, a guide wire of alternative shape. FIG. 5 shows a loop, spiral or pig-tail shaped guide wire 25 that may be used to form a line of lesion around or along the renal component. Although shown as a spiral or pigtail shape 25, any other suitable shape may also be used to form the line of irradiation, and the present invention is not limited in its broader aspects to the particular guide wire shape. The shape of the irradiation line or point may be also be predetermined by imparting features to the source train delivery catheter with or without a shape-modifying guide wire, insert or stylus. A guide wire 21, stylus or other insert, removable or fixed type, may also be used to create preferred catheter 19 geometry to facilitate vascular site access, facilitate accuracy of delivery catheter and source positioning with or without radiopaque or other imaging features as well as functions to test catheter patency and navigability for source delivery.

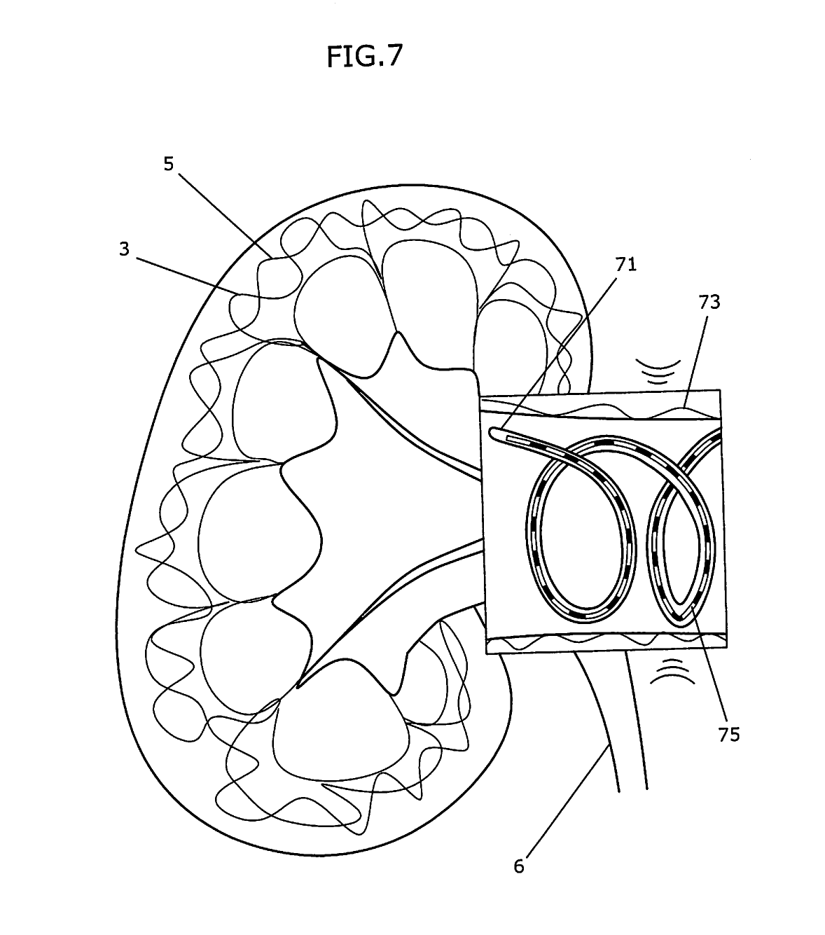

FIG. 7 illustrates a close-up of the guide catheter 71 with the source train 75. Specifically, the renal wall contains the renal nerves 73. The pig-tail guide catheter 71 allows for a helical shape in the renal vessel, which directly results in maximum circular radiation. The pig-tail shape is in a helical shape throughout the entire vessel for the maximum coverage; however, there may be partial insertion depending on a patient's condition or depending on the anatomical structure of the renal vessel.

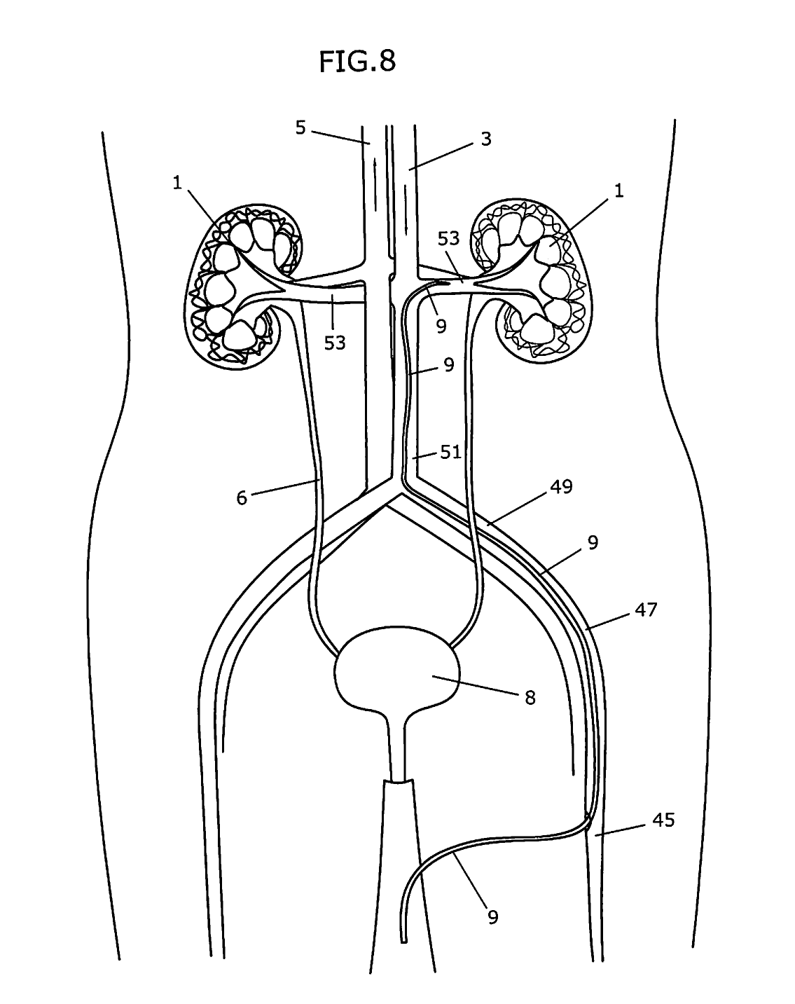

FIG. 8 illustrates one of many different entry points into the patient for treatment; specifically, one means of entry into the renal vein is into the vena cava; preferably, starting with the femoral vein 45 through the external iliac vein 47 and then all the way to the common iliac vein 49 up to the abdominal vena cava 51 and inside the left and right renal vein 53; of course, other approaches to the renal vein may be used without departing from the present invention. For treating renal irradiation, the guide wire or catheter may be shaped to form a continuous lesion in the renal wall, and or isolating the renal vein(s) from the remainder of the left renal artery. Other radiosensitive organs, like the ureters 6 and kidneys 1, will be protected from unnecessary radiation by limiting the penetrating range, energy or fluence by choice or spectrum of radionuclide, particles or photons, interposing radiation attenuating materials or filters and geometry by catheter or guidewire shapes and features.

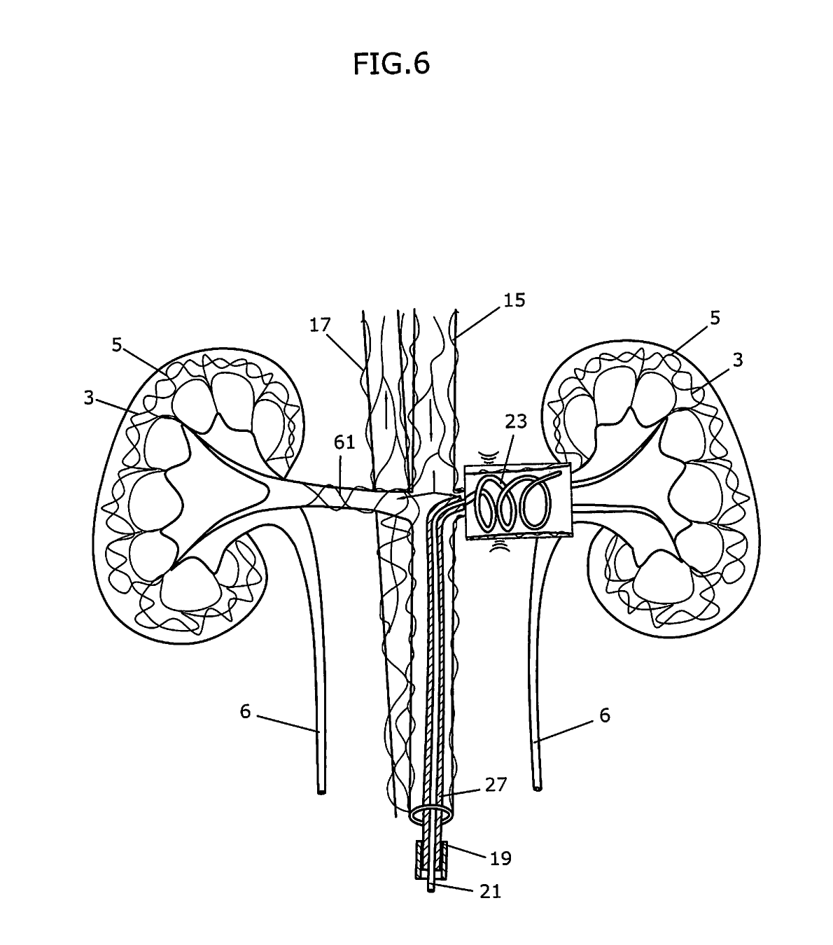

In FIG. 4 and FIG. 6, the straight point or pigtail shape is particularly useful for locating the radiation source catheter within a renal vessel itself and treating renal vessel irradiation by exposing the surface of the renal vessel to a dose of ionizing radiation. Alternatively, the guide wire 21 portion located in the renal vessel could be straight forward and centrally located within the vessel.

It is contemplated that these alternative shapes would be used with a radiation source delivery catheter having a sufficiently flexible distal end to conform to the shape of the guide wire or vessel. The guide wires 21 of FIG. 4 and FIG. 6 could be of any suitable material, such as stainless steel, titanium or nickel-titanium alloy.

Alternatively, the catheter 19 itself could have a pre-shaped distal end, such as curved, pig-tail or spiral to engage the blood vessel wall in the desired position for irradiation. This shape could be set into the end of the catheter using known techniques such as heat setting, molding, balloons or the like. With this type of catheter, the guide wire 21 would tend to straighten the catheter during insertion, and withdrawal of the guide wire would allow the catheter to resume its preset shape. After properly positioning the guide wire 21 and catheter 19 against the wall of the renal vein or artery at the location to be irradiated, the radioactive sources would be inserted into the end of the catheter for the irradiation treatment.

The catheter, in FIG. 4 may also have electrode(s) or sensor(s) 41, such as bipolar, carried at the distal end portion and communicating via conductors extending through the catheter to a proximal location outside the patient's body. At least one such electrode is contemplated, and preferably at least two electrodes or sensors, such as one proximal to the radiation source and one distal to the radiation source. Two electrodes or sensors would allow sensing of conductivity across a line of lesion to determine if nerve conduction is complete. Also, the electrodes would allow for direct sensing and monitoring or electrophysiological characteristics of the kidney tissue before, during and/or after ablation and well mapping the electrophysiology of the kidney to determine the appropriate site for irradiation treatment. The electrodes would be connected through one or more conductors extending through the catheter to a monitoring or readout device located outside the patient's body.

Further, the catheter may include a cooling surface 43 on the distal end portion for cooling selected renal tissue, for example, to identify the desired site for irradiation treatment. This cooling surface could be based on the Peltier effect, as disclosed for example in the previously mentioned U.S. Pat. No. 5,529,067, and also connected via one or more conductors extending through the catheter. More specifically, systematic cooling of selected renal tissue and observation of the effect of cooling on the electrophysiology may be used to identify the location of tissue to be treated, and once identified, the treatment can be immediately carried out by advancing the radiation source through the catheter and to the site without further movement of the catheter required. This has the potential benefit of better assuring that treatment is being carried out at the desired location.

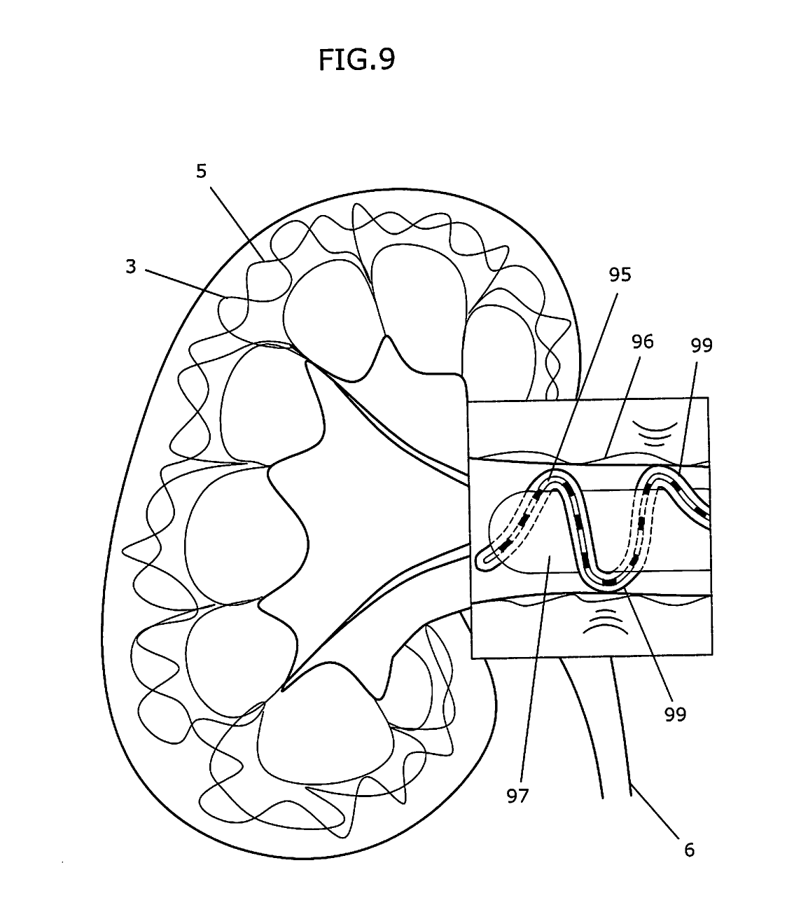

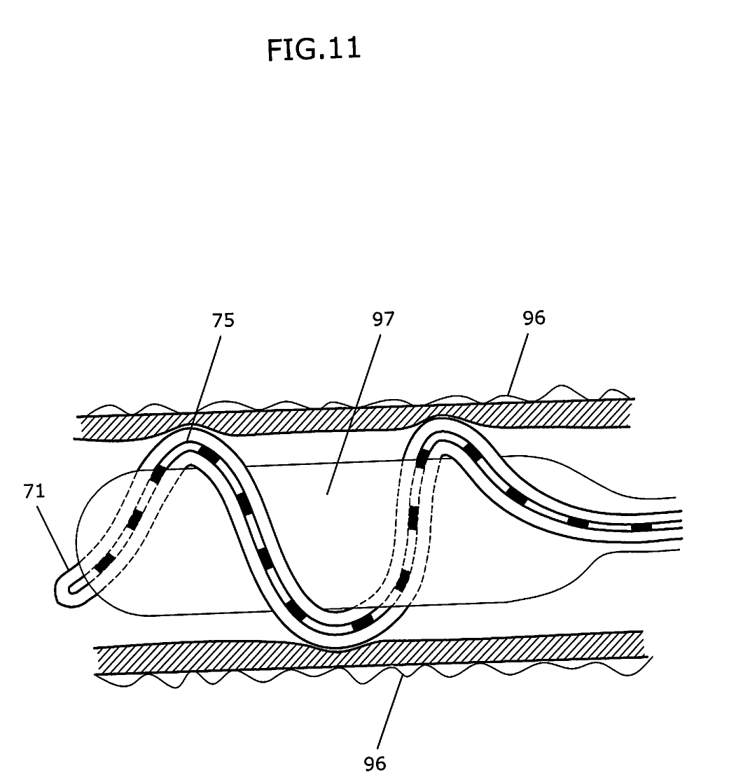

FIG. 9 illustrates an overview of the human kidney with left side irradiated with pigtail shape catheter 99 expanded with an inflator 97. The inflator 97 is used to expand the pigtail shape catheter 99 to the renal walls. The inflator benefits the patient since the source train pushes closer to the walls of the renal vessels, and further, allows for a tight fit for the guide catheter. In addition, the expanded inflator allows for source train 95 to move easier to the end of the guide catheter 99. The expansion of the inflator 97 is further illustrated in FIG. 11. The inflator 97 can provide benefit to the patient since all vessels in the human body are not perfectly cylindrical. The vessels may be deformed or clogged with deposits of minerals or fat. This creates a problem when trying to insert the guide wire. However, when a guide wire is inserted into a narrow vessel; the catheter 71 may not be inserted into the vessel due to obstruction. By have an inflator 97, medical personnel are able to expand the size of the vessel and insert the catheter 71 with the radiation source 75. Furthermore, with deposits inside the vessel, the expansion of the inflator 97 allows for the catheter 71 and source train 75 to be closer to the renal nerves 96. The inflator 97 not only expands the vessel walls but may also extend the length of the catheter 75 out further. This provides a benefit to when the vessel is partially obstructed for the medical personnel to irradiate the renal nerves conformally.

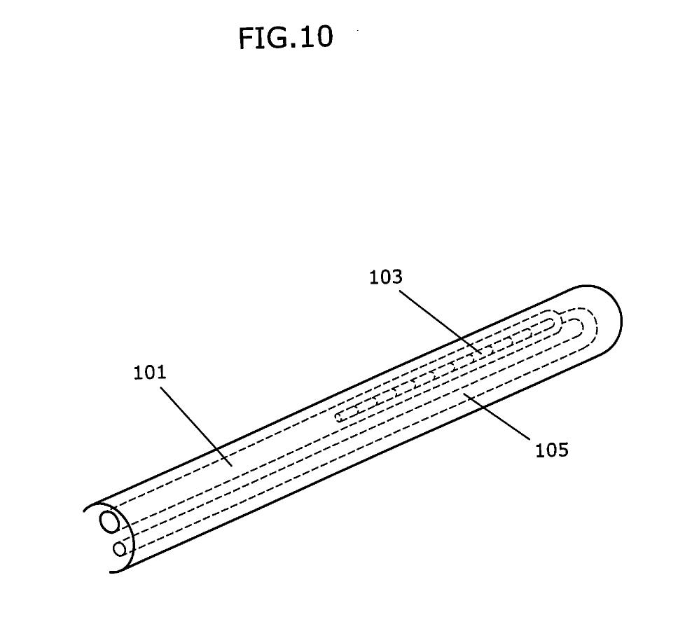

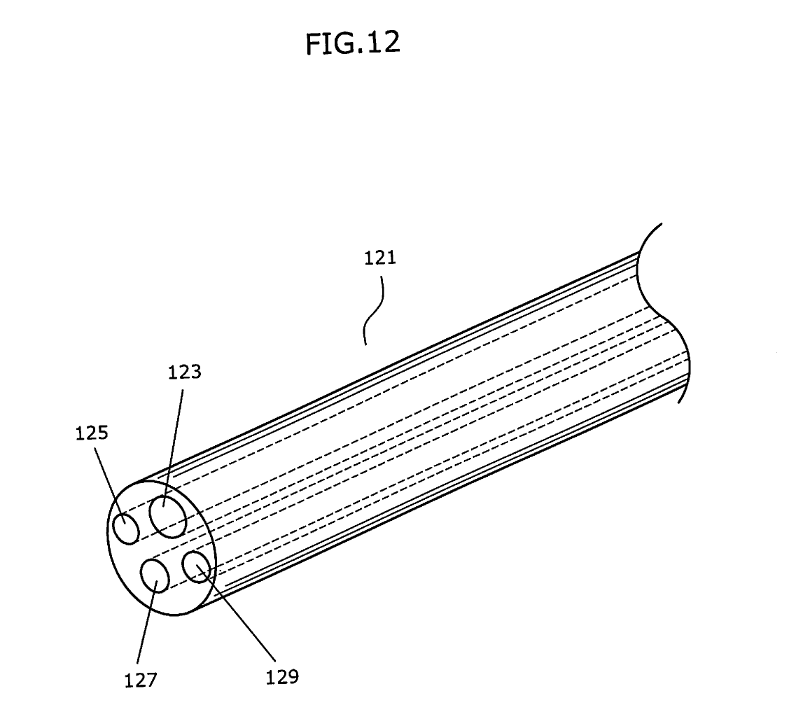

FIG. 10 illustrates a cross-sectional guide catheter with directional lumens. Specifically, the guide catheter contains two specific lumens. The source train lumen 101, which is used to hold the radioactive sources 103 and moves the source train to the tip of the catheter. The source train is inserted into [prior steps to use the device]. The source train is pumped up the lumen to the distal end of the guide catheter by a hydraulic pump. The hydraulic pump has hydraulic pump return lumen 105, which returns the liquid back into the device. FIG. 12 illustrates configuration of the guide catheter, but may not be used to limit the invention to specific constraints. In addition, the guide catheter also contains a geometric control of the treatment zone 125. This is used to increase or decrease the inflator depending on the patient's usage. Also, the guide catheter contains a guide wire lumen 127. The guide wire lumen is used to place the guide catheter to the location of the lumen.

1. A method of irradiating a plurality of renal nerves comprising:
puncturing a human body to insert a guidewire to a region of interest;
connecting said guidewire to an elongated catheter distal end portion into the circulatory system of said human body;
inserting said elongated catheter into said human body until said elongated catheter distal end is located near a renal vessel;
adjusting said elongated catheter distal end near a tissue wall of said renal vessel,
wherein, said tissue wall of said renal vessel is in close proximity to said plurality of renal nerves;
moving a source train through a lumen within said elongated catheter until said source train reaches said renal vessel;
irradiating said plurality of renal nerves by holding said source train for a predetermined time interval in said renal vessel; and
removing said source train from the renal vessel after a lapsed in said predetermined time interval.

2. A method of claim 1, wherein said guidewire can be shaped comprises a straight distal end, a pigtail shape distal end, or a half circle distal end.

3. A method of claim 2, wherein said elongated catheter distal end may expand or shrink; the method further comprising inflating an inflator by inserting gas or liquid into a inflator lumen.

4. An apparatus for irradiating a plurality of renal nerves comprising:
a guidewire used to lead an elongated catheter in the human body;
said enlongated catheter further comprising a plurality of lumens;
said plurality of lumens further comprising a source train lumen, a hydraulic pump lumen, a guidewire lumen, and an inflator lumen;
said elongated catheter is connected to said guidewire by inserting said guidewire into said guidewire lumen;
said elongated catheter contains a source train within said source train lumen;
said source train lumen and hydraulic pump lumen are used to push said source train to a renal vessel.

5. The apparatus for irradiating said plurality of renal nerves comprising:
said elongated catheter at said distal end further comprising an inflator, wherein said inflator is connected to said inflator lumen and said inflator lumen can expand and contract depending on the fill volume.

6. The apparatus for irradiating said plurality of renal nerves comprising:
said inflator at said distal tip of said elongated catheter is located inside the wrapped said elongated catheter; wherein, said elongated catheter can expand to the renal vessel walls.
